(19) United States
(12) Patent Application Publication
(10) Pub. No.: US 2025/0128004 A1
(43) Pub. Date: Apr. 24, 2025

(54) BLOOD FLOW CONTROL APPARATUS AND METHODS

(71) Applicant: UTI Limited Partnership, Calgary (CA)

(72) Inventors: Aaron PHILLIPS, Calgary (CA); Kelly KAISER, Calgary (CA); Ryan Raymond MILLER, Dunmore (CA); Tyler LAM, Calgary (CA)

(21) Appl. No.: 18/856,058

(22) PCT Filed: Jan. 25, 2023
(86) PCT No.: PCT/CA2023/050093

§ 371 Date: Oct. 11, 2024

Related U.S. Application Data
(60) Provisional application No. 63/267,121, filed on Jan. 25, 2022.

(51) Int. Cl. A61M 16/00; A61M 5/172; A61M 16/12

(52) U.S. Cl. CPC A61M 16/022; A61M 5/1723; A61M 16/12; A61M 2202/0225; A61M 2205/3313; A61M 2205/3334; A61M 2205/3344; A61M 2205/3375

(57) ABSTRACT

An apparatus and methods are provided that operate to stabilize blood flow at the site of an injury in a patient, particularly in tissues of the central nervous system. Such an apparatus and methods may mitigate the severity of an injury by optimizing blood flow and reducing secondary damage, leading to improved neurological recovery. A closed-loop system may control one or more parameters at the site of injury by regulating circulating carbon dioxide levels or carbon dioxide and oxygen and/or pH. An example embodiment includes a controller that controls a gas mixer to vary the CO₂ concentration in a gas supplied for breathing by a patient in response to an output signal from a sensor that monitors one or more desired outcomes. Example applications of the present technology include treating spinal cord injury, traumatic brain injury, and cardiac condition or event.

### REFERENCE TO RELATED APPLICATIONS

This application claims priority from, and for the purposes of the United States the benefit under 35 USC 119 in connection with, U.S. patent application No. 63/267,121 filed 25 Jan. 2022, which is hereby incorporated herein by reference.

### FIELD

This invention relates to control of hemodynamics such as blood flow and/or blood pressure. Example embodiments of the invention have application in controlling blood flow and/or blood pressure in persons suffering from injuries to the central nervous system (CNS) such as spinal cord injury (SCI), traumatic brain injury (TBI), and any other conditions that may lead to compromised CNS blood flow, as well as cardiac conditions, such as myocardial infarction, angina, heart failure and/or the like.

### BACKGROUND

Injuries to the central nervous system such as spinal cord injury and traumatic brain injury (TBI) as well as injuries from cardiac conditions or acute cardiac events can have devastating consequences including sensory, motor, and autonomic deficits. Consequences of such injuries can significantly impair overall quality of life. In the acute phase (within hours to days) of a CNS injury, the site of injury can display ischemia, hypoxia, and reduced blood flow in the penumbra of the injury which leads to secondary damage. Secondary damage has been shown to contribute to a significant portion of the final injury and lasting outcomes. Cardiac conditions (including, but not limited to, acute cardiac events) can lead to similar secondary effects.

Preventing secondary damage has the potential to significantly reduce the severity of CNS injuries and/or cardiac conditions. However, it has proved to be challenging to find practical treatments that reduce secondary damage and improve outcomes in patients suffering from CNS injuries and/or cardiac conditions. Current approaches are limited in their effectiveness.

References that provide background information that may assist in understanding the present invention include:
1. Anjum, A. et al. Spinal Cord Injury: Pathophysiology, Multimolecular Interactions, and Underlying Recovery Mechanisms. Int. J. Mol. Sci. 21, (2020).
2. Alizadeh, A., Dyck, S. M. & Karimi-Abdolrezaee, S. Traumatic Spinal Cord Injury: An Overview of Pathophysiology, Models and Acute Injury Mechanisms. Front. Neurol. 10, 282 (2019).
3. Squair, J. W. et al. Neuroprosthetic baroreflex controls haemodynamics after spinal cord injury. Nature 1-7 (2021).
4. Ahuja, C. S. et al. Traumatic Spinal Cord Injury-Repair and Regeneration. Neurosurgery 80, S9-S22 (2017).
5. Evaniew, N., Mazlouman, S. J., Belley-Côté, E. P., Jacobs, W. B. & Kwon, B. K. Interventions to Optimize Spinal Cord Perfusion in Patients with Acute Traumatic Spinal Cord Injuries: A Systematic Review. J. Neurotrauma 37, 1127-1139 (2020).
6. Lee, Y.-S., Kim, K.-T. & Kwon, B. K. Hemodynamic Management of Acute Spinal Cord Injury: A Literature Review. Neurospine 18, 7-14 (2021).
7. Li, Y. et al. Pericytes impair capillary blood flow and motor function after chronic spinal cord injury. Nat. Med. 23, 733-741 (2017).
8. Rowland, J. W., Hawryluk, G. W. J., Kwon, B. & Fehlings, M. G. Current status of acute spinal cord injury pathophysiology and emerging therapies: promise on the horizon. Neurosurg. Focus 25, E2 (2008).
9. Yuan, X. et al. Exosomes Derived From Pericytes Improve Microcirculation and Protect Blood—Spinal Cord Barrier After Spinal Cord Injury in Mice. Front. Neurosci. 13, 319 (2019).

There is a need for apparatus and methods that are practical and easily operable to potentially improve outcomes in people suffering from CNS injuries and/or cardiac conditions.

### SUMMARY

This invention has a number of aspects. These include, without limitation:
apparatus for controlling blood flow and/or blood pressure, particularly in the CNS (e.g. spinal cord or brain), particularly near the site of an SCI or brain injury or, in the case of a cardiac condition, in the region of the heart and/or the coronary arteries;
methods for controlling blood flow and/or blood pressure, particularly in the CNS (e.g. spinal cord or brain), particularly near the site of an SCI or brain injury or, in the case of a cardiac condition, in the region of the heart and/or the coronary arteries;
apparatus for providing feedback control of circulating carbonic anhydrase levels (e.g. carbonic anhydrase levels in the circulatory system and/or otherwise circulating or present in the body of a patient) based on measurements of blood flow and/or blood pressure and/or blood pH level, particularly in the CNS (e.g. spinal cord or brain), particularly near the site of an SCI or brain injury or, in the case of a cardiac condition, in the region of the heart and/or the coronary arteries;
methods for providing feedback control of circulating carbonic anhydrase levels based on measurements of blood flow and/or blood pressure and/or blood pH level, particularly in the CNS (e.g. spinal cord or brain), particularly near the site of an SCI or brain injury or, in the case of a cardiac condition, in the region of the heart and/or the coronary arteries;
apparatus for providing feedback control of an inspired gas and/or an administered substance based on measurements of blood flow and/or blood pressure and/or blood pH level, particularly in the CNS (e.g. spinal cord or brain), particularly near the site of an SCI or brain injury or, in the case of a cardiac condition, in the region of the heart and/or the coronary arteries;
methods for providing feedback control of an inspired gas and/or an administered substance based on measurements of blood flow and/or blood pressure and/or blood pH level, particularly in the CNS (e.g. spinal cord or brain), particularly near the site of an SCI or brain injury or, in the case of a cardiac condition, in the region of the heart and/or the coronary arteries;
apparatus for providing feedback control of blood gas levels based on measurements of blood flow and/or blood pressure and/or blood pH level, particularly in the CNS (e.g. spinal cord or brain), particularly near the site of an SCI or brain injury or, in the case of a cardiac condition, in the region of the heart and/or the coronary arteries;
methods for providing feedback control of blood gas levels based on measurements of blood flow and/or blood pressure and/or blood pH level particularly in the CNS (e.g. spinal cord or brain), particularly near the site of an SCI or brain injury or, in the case of a cardiac condition, in the region of the heart and/or the coronary arteries; and/or
combinations and sub-combinations of the above.

One aspect of the invention provides an apparatus for stabilizing blood flow, the apparatus comprising: a pH-adjusting actuator operative to deliver a pH-modifying agent to a patient; a controller connected to control the pH-adjusting actuator to alter a level at which the pH-modifying agent is delivered to the patient in response to an output signal from a sensor, the sensor operative to monitor blood flow; wherein the controller is configured to cause the pH-adjusting actuator to modify the level at which the pH-modifying agent is delivered to the patient in a manner which causes the blood pH in the patient to decrease in response to the sensor output signal indicating a decrease in the blood flow.

The pH-adjusting actuator may comprise a gas source and the pH modifying agent comprises CO₂ such that the gas source is operative to deliver CO₂ to the patient. The controller may be configured to increase the level at which the CO₂ is delivered to the patient in response to the sensor output signal indicating a decrease in the blood flow.

The gas source may comprise a gas mixer operative to output a mixed gas, a source of CO₂ and a source of other breathable gases. The controller may be operable to control the gas source by controlling the gas mixer to vary a ratio of CO₂ from the source of CO₂ to other breathable gases from the source of other breathable gases in the mixed gas and the apparatus comprises an interface configured to supply the mixed gas to the patient for breathing.

The controller may be configured to: average the output signal from the sensor over a time period; process the averaged output signal to determine whether the blood flow is below, within, or above an optimal range; in response to the blood flow being below the optimal range increase the level at which the CO2 is delivered until the blood flow is within the optimal range; and in response to the blood flow being above the optimal range decrease the level at which the CO2 is delivered until the blood flow is within the optimal range.

The controller may be configured to increase the level at which the CO₂ is delivered by incrementally increasing the level and pausing between increments.

The pH-adjusting actuator may comprise a drug-delivery system and the pH-modifying agent may comprise a pH-modifying drug, the drug-delivery system operable to deliver the pH-modifying drug to the patient. The pH-modifying drug may comprise a carbonic anhydrase inhibiting drug. The carbonic anhydrase inhibiting drug comprises at least one of acetazolamide.

The controller may be configured to increase a dosage at which the pH modifying drug is delivered to the patient in response to the sensor output signal indicating a decrease in the blood flow.

The drug-delivery system may comprise a drug-delivery pump and/or a drug-eluding device.

The controller may be configured to: average the output signal from the sensor over a time period; process the averaged output signal to determine whether the blood flow is below, within, or above an optimal range; in response to the blood flow being below the optimal range increase the level at which the pH-modifying drug is delivered until the blood flow is within the optimal range; and in response to the blood flow being above the optimal range decrease the level at which the pH-modifying drug is delivered until the blood flow is within the optimal range.

The controller may be configured to increase the level at which the pH-modifying drug is delivered by incrementally increasing the level and pausing between increments.

The sensor may comprises a Doppler ultrasound blood flow sensor and/or a near infrared sensor. The sensor may be operational to monitor at least one of: spinal cord perfusion pressure and coronary perfusion pressure.

The controller may be configured to execute a feedback control algorithm.

Another aspect of the invention provides a controller for stabilizing blood flow, the controller comprising: an input for receiving a sensor output signal indicative of blood flow in a patient; an output for outputting a control signal for a pH-adjusting actuator operative to deliver a pH-modifying agent to a patient; and a processor configured to execute a feedback control algorithm, the feedback control algorithm operative to, in response to the blood flow being lower than an optimal range, set the control signal to cause the pH-adjusting actuator to modify an output level of the pH-modifying agent for delivery to the patient.

The pH-adjusting actuator may comprise a gas source and the pH-modifying agent may comprise CO₂, the gas source operative to deliver CO₂ to the patient. The controller may be configured to set the control signal to cause the gas source to increase the output level of CO₂ for delivery to the patient in response to the blood flow being lower than an optimal range.

The pH-adjusting actuator may comprise a gas mixer and the pH-modifying agent may comprise CO₂, the gas mixer operative to deliver CO₂ to the patient.

The controller may be configured to set the control signal to cause the gas mixer to increase the output level of CO₂ for delivery to the patient in response to the blood flow being lower than an optimal range.

The pH-adjusting actuator may comprise a drug-delivery system and the pH-modifying agent comprises a pH-modifying drug, the drug-delivery system operable to deliver the pH-modifying drug to the patient.

The controller may be configured to set the control signal to cause the drug-delivery system to increase a dosage of the pH-modifying drug for delivery to the patient in response to the blood flow being lower than an optimal range. Another aspect of the invention provides a method for stabilizing blood flow, the method comprising the steps of: measuring a value of an input parameter indicative of blood flow in a patient; assessing whether the value of the input parameter is below, within or above an optimal range of a characteristic of interest; and adjusting a level at which a pH-modifying agent is delivered to the patient based at least on the assessment of the value of the input parameter relative to the optimal range of the characteristic of interest.

The value of the input parameter may be averaged over a time period.

The method may comprise outputting a control signal to control the level at which the pH-modifying agent is delivered to the patient.

The method may comprise executing a feedback control algorithm, the feedback control algorithm operative to, in response to the characteristic of interest being lower than an optimal range, set the control signal to cause an increase in output level of the pH-modifying agent for delivery to the patient.

The pH-modifying agent may comprise CO₂ gas and/or a pH-modifying drug. The pH modifying drug may comprise a carbonic anhydrase inhibitor.

Another aspect of the invention provides an apparatus for stabilizing blood flow, the apparatus comprising: a gas source operative to deliver CO2 to a patient; a controller connected to control the gas source to alter a level at which the CO2 is delivered to the patient in response to an output signal from a sensor, the sensor operative to monitor blood flow; wherein the controller is configured to increase the level at which the CO2 is delivered to the patient in response to the sensor output signal indicating a decrease in the blood flow.

The gas source may comprise a gas mixer operative to output a mixed gas, a source of CO2 and a source of other breathable gases. The controller may be operable to control the gas source by controlling the gas mixer to vary a ratio of CO2 from the source of CO2 to other breathable gases from the source of other breathable gases in the mixed gas. The apparatus may comprise an interface configured to supply the mixed gas to the patient for breathing.

The sensor may comprises a Doppler ultrasound blood flow sensor. The sensor may be operational to monitor at least one of: spinal cord perfusion pressure; and coronary perfusion pressure. The sensor may comprise a near infrared sensor.

The controller may be configured to execute a feedback control algorithm.

The controller may be configured to: average the output signal from the sensor over a time period; process the averaged output signal to determine whether the blood flow is below, within, or above an optimal range; in response to the blood flow being below the optimal range increase the level at which the CO2 is delivered until the blood flow is within the optimal range; and in response to the blood flow being above the optimal range decrease the level at which the CO2 is delivered until the blood flow is within the optimal range.

The controller may be configured to increase the level at which the CO₂ is delivered by incrementally increasing the level and pausing between increments.

Another aspect of the invention provides a controller for stabilizing blood flow, the controller comprising: an input for receiving a sensor output signal indicative of blood flow in a patient; an output for outputting a control signal for a gas mixer; and a processor configured to execute a feedback control algorithm, the feedback control algorithm operative to, in response to the blood flow being lower than an optimal range, set the control signal to cause the gas mixer to increase an output level of CO2 for delivery to a patient.

Another aspect of the invention provides an apparatus for stabilizing blood flow, the apparatus comprising: a drug delivery system operative to deliver a vascular impacting drug to a patient; a controller connected to control the drug delivery system to alter a level at which the vascular-impacting drug is delivered to the patient in response to an output signal from a sensor, the sensor operative to monitor blood flow; wherein the controller is configured to cause the drug delivery system to modify the level at which the vascular-impacting drug is delivered to the patient in a manner which causes the dilation of at least one blood vessel of the patient in response to the sensor output signal indicating a decrease in the blood flow.

The sensor may comprises a Doppler ultrasound blood flow sensor. The sensor may be operational to monitor at least one of: spinal cord perfusion pressure; and coronary perfusion pressure. The sensor may comprise a near infrared sensor.

The controller may be configured to execute a feedback control algorithm. The controller may be configured to: average the output signal from the sensor over a time period; process the averaged output signal to determine whether the blood flow is below, within, or above an optimal range; in response to the blood flow being below the optimal range increase the level at which the vascular-impacting drug is delivered until the blood flow is within the optimal range; and in response to the blood flow being above the optimal range decrease the level at which the vascular-impacting drug is delivered until the blood flow is within the optimal range.

The controller may be configured to increase the level at which the vascular-impacting drug is delivered by incrementally increasing the level and pausing between increments.

Another aspect of the invention provides a controller for stabilizing blood flow, the controller comprising: an input for receiving a sensor output signal indicative of blood flow in a patient; an output for outputting a control signal for a drug-delivery system; and a processor configured to execute a feedback control algorithm, the feedback control algorithm operative to, in response to the blood flow being lower than an optimal range, set the control signal to cause the drug-delivery system to increase an output level of a vascular-impacting drug for delivery to a patient.

Other aspects of the invention provide apparatus having any new and inventive feature, combination of features, or sub-combination of features as described herein.

Other aspects of the invention provide methods having any new and inventive steps, acts, combination of steps and/or acts or sub-combination of steps and/or acts as described herein.

Further aspects and example embodiments are illustrated in the accompanying drawings and/or described in the following description.

It is emphasized that the invention relates to all combinations of the above features, even if these are recited in different claims, illustrated in different drawings and/or described in different sections, paragraphs and/or sentences.

### BRIEF DESCRIPTION OF THE DRAWINGS

The accompanying drawings illustrate non-limiting example embodiments of the invention.

FIG. 1A is a block diagram of an apparatus according to an example embodiment.

FIG. 1B is a block diagram of an apparatus according to another example embodiment.

FIG. 1C is a block diagram of an apparatus according to another example embodiment.

FIG. 2 is a flow chart illustrating a method for operating the FIG. 1B apparatus according to an example embodiment.

FIG. 3 is a schematic view of a controller illustrating a selection of control inputs and monitor functions.

FIGS. 4A-F include various plots showing the correlation between circulating carbon dioxide levels and various physiological parameters in an experimental setting.

FIGS. 5 is a plot tracing blood flow of uninjured test animals, injured test animals and injured test animals treated by the method in FIG. 2 over a period of time in an experimental setting.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Some aspects of the invention exploit the fact that hemodynamics (e.g. blood flow and/or blood pressure) in particular regions of the body (e.g. in the central nervous system (CNS) and/or in the heart and/or coronary arteries are strongly influenced by pH, with decreasing pH associated with increases in blood flow/blood pressure and increasing pH being associated with decreases in blood flow.

One naturally occurring reaction within the body is interconversion (shown by equation (1) below) between carbon dioxide and water and the dissociated ions of carbonic acid (i.e. bicarbonate and hydrogen ions).

CO₂+H₂O<=>CO₃H₂<=>CO₃H⁻+H⁺ (1)

Typically, in equation (1), the reaction CO₂+H₂O<=>CO₃H₂ is a reversible enzymatic reaction catalyzed by carbonic anhydrase and the reaction CO₃H₂<=>CO₃H⁻+H⁺ is a spontaneous non-enzymatic dissociation.

Some aspects of the present technology exploit the fact that the pH in particular regions of the body (e.g. in the central nervous system (CNS) and/or in the heart and/or coronary arteries) is strongly influenced by circulating carbon dioxide levels (e.g. carbon dioxide levels in the circulatory system of a patient and/or otherwise circulating or present in the body of a patient). One measure of circulating carbon dioxide levels is the arterial partial pressure of carbon dioxide (Pa_{CO2}). It will be appreciated from equation (1) that with other things being equal, increasing Pa_{CO2} will drive equation (1) to the right, thereby releasing more hydrogen ions and correspondingly decreasing the pH. As discussed above, decreasing pH in particular regions of the body causes dilation of local vessels, thereby increasing blood flow and decreasing blood pressure in such regions.

Some aspects of the present technology exploits the fact that the pH in particular regions of the body (e.g. in the central nervous system (CNS) and/or in the heart and/or coronary arteries) is additionally or alternatively influenced by circulating carbonic anhydrase levels. As alluded to above carbonic anhydrases are enzymes that catalyze the equation (1) interconversion of CO₂+H₂O<=>CO₃H₂. It will be appreciated from equation (1) that reducing circulating carbonic anhydrase levels (e.g. concentration) reduces the rate of interconversion of CO₂+H₂O<=>CO₃H₂ and, effectively lowers the pH by preventing the accumulation of H+ ions in the body. That is H+ ions accumulate naturally in the body, but equation (1) is prevented from being driven leftward by the lack of available carbonic anhydrase to act as a catalyst. As discussed above, decreasing pH in particular regions of the body causes dilation of local vessels, thereby increasing blood flow and decreasing blood pressure in such regions.

In an example method, the CNS region or cardiac region (e.g. heart and/or coronary artery) blood flow and/or blood pressure are monitored in a patient. Based on the results of the monitoring, the pH is adjusted in CNS region or in the cardiac region to alter the CNS region or cardiac region blood flow and/or blood pressure in such a way that it causes the patients CNS region or cardiac region blood flow and/or blood pressure to change in a way that brings the patient's CNS region or cardiac region blood flow and/or blood pressure toward a desired value. In some embodiments, the pH in the CNS region or cardiac region is adjusted by adjusting levels of circulating carbon dioxide and/or circulating carbonic anhydrase. to alter the central nervous system or cardiac region blood flow and/or blood pressure in such a way that it causes the patient's central nervous system or cardiac region blood flow and/or blood pressure to change in a way that brings the patient's central nervous system or cardiac region blood flow and/or blood pressure toward a desired value. For example:
if CNS/cardiac regional blood flow is lower than desired, the blood pH may be reduced, since decreasing pH tends to increase CNS/cardiac region blood flow;
if CNS/cardiac regional blood flow is greater than desired, the blood pH may be increased, since increasing pH tends to decrease CNS/cardiac region blood flow;
if CNS/cardiac regional blood flow is within a desired range, the blood pH may be maintained;
if CNS/cardiac regional blood flow is lower than desired, the circulating carbon dioxide levels may be increased, since an increase in circulating carbon dioxide levels tends to lower the PH and thereby increase CNS/cardiac region blood flow and/or since an increase in circulating carbon dioxide levels tend to increase vascular dilation and thereby increase CNS/cardiac region blood flow;
if CNS/cardiac regional blood flow is greater than desired the circulating carbon dioxide levels may be decreased, since a decrease in circulating carbon dioxide levels tends to increase the pH and thereby decrease CNS/cardiac region blood flow and/or since an decrease in circulating carbon dioxide levels tend to decrease vascular dilation and thereby decrease CNS/cardiac region blood flow;
if CNS/cardiac regional blood flow is within a desired range the circulating carbon dioxide levels may be maintained;
if CNS/cardiac regional blood flow is lower than desired, the circulating carbonic anhydrase levels may be decreased (e.g. by changing a dosage of carbonic anhydrase inhibiting drugs), since a decrease in circulating carbonic anhydrase levels tends to lower the pH and thereby increase CNS/cardiac region blood flow;
if CNS/cardiac regional blood flow is greater than desired, the circulating carbonic anhydrase levels may be increased (e.g. by changing a dosage of carbonic anhydrase inhibiting drugs), since an increase in circulating carbonic anhydrase levels tends increase the pH and thereby decrease CNS/cardiac region blood flow; and
if CNS/cardiac regional blood flow is within a desired range the circulating carbonic anhydrase levels may be maintained (e.g. by maintaining a dosage of carbonic anhydrase inhibiting drugs).

Circulating carbon dioxide levels may be increased (and blood pH may be decreased), for example, by:
increasing a concentration, or Pa_{CO2}, in a gas being inspired by the patient;
directly introducing CO₂ or a chemical species (such as carbonate or bicarbonate) that is in equilibrium with CO₂ into the patient's blood.

Circulating carbonic anhydrase levels may be decreased (and pH may be decreased), for example, by:
increasing dosage of a carbonic anhydrase inhibiting drug, such as acetazolamide and/or the like (e.g. via an implantable or external drug delivery pump, a drug eluding array, IV infusion, a drug eluding stent and/or the like).
   Similar delivery systems may be used for other types of drugs (e.g. vascular-impacting drugs, such as sodium nitroprusside and/or the like).

In general, other suitable additional or alternative techniques could be used to alter the local blood pH in a CNS/cardiac region and to thereby assist in controlling blood flow and/or blood pressure in the CNS/cardiac region. Other blood levels that may be controlled in conjunction with pH levels, carbon dioxide levels and/or carbonic anhydrase levels to assist in controlling blood flow and/or blood pressure and/or to assist in improving outcomes include oxygen levels (e.g. Pa_{O2}).

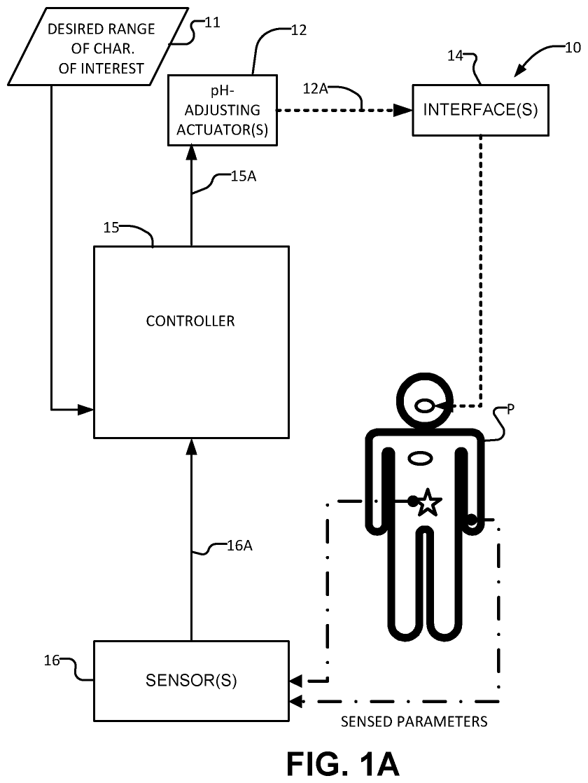

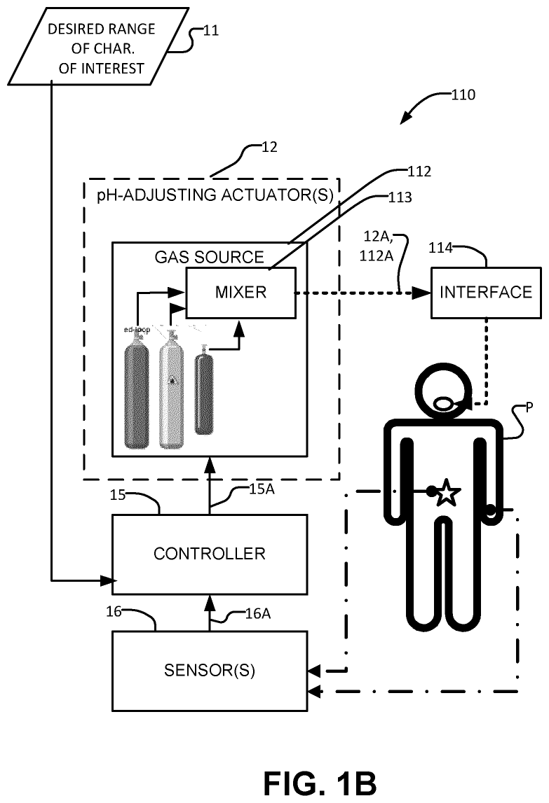

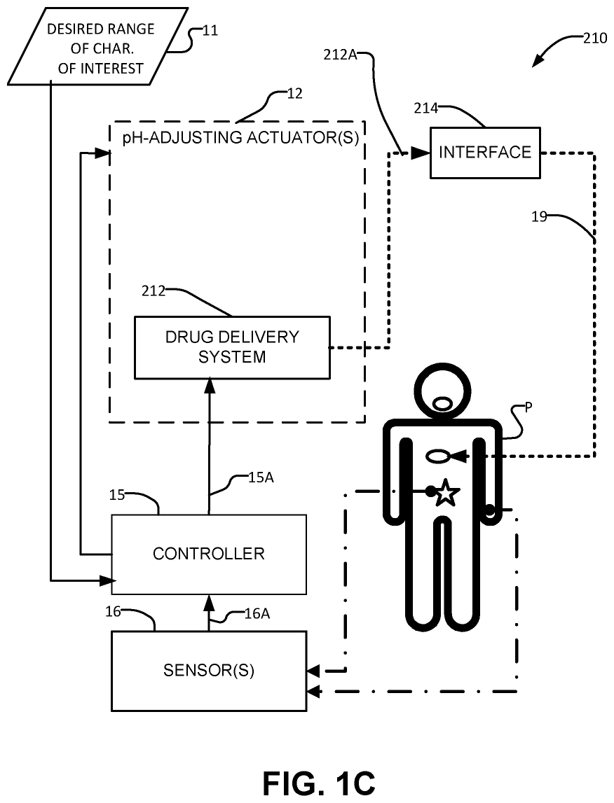

FIG. 1A illustrates a blood flow control apparatus 10 according to an example embodiment of the invention. Apparatus 10 includes one or more pH-adjusting actuator(s) 12 (which may include, for example, a gas source and/or a gas mixer for adjusting the blood pH in patient P using carbon dioxide and/or a drug delivery system for adjusting the blood pH in patient P using a drug and/or some other agent for inhibiting the blood concentration of carbonic anhydrase), an interface 14 for introducing the output 12A (e.g. a pH-adjusting agent) of pH-adjusting actuator(s) 12 to patient P, a controller 15 operative to control actuator(s) 12 and one or more sensors 16 to monitor physiological characteristics of patient P.

Controller 15 provides closed loop control of a characteristic of interest to control the characteristic of interest to be at or around a desired level or within a desired range. The desired range of a characteristic of interest 11 may be provided as an input (e.g. via a suitable user interface) to controller 15. In currently preferred embodiments, the characteristic of interest comprises a parameter related to CNS/cardiac blood flow and/or CNS/cardiac blood pressure. Controller effects this closed loop control by using feedback from sensors 16 to generate control signals 15A which are in turn provided to pH-adjusting actuator(s) 12 to cause actuator(s) 12 to administer a pH-modifying agent 12A to patient P via interface 14. Controller 15 may use control signals 15A to modify a dosage (e.g. an amount, a constitution and/or the like) of pH-modifying agent 12A that is administered to patient P.

FIG. 1B illustrates a blood flow control apparatus 110 according to another example embodiment of the invention. Apparatus 110 differs from apparatus 10 described above in that pH-adjusting actuator(s) 12 of apparatus 110 comprise a gas source 112 and, optionally, a gas mixer 113. Gas source 112 and/or gas mixer 113 are controlled by controller 15 (using control signals 15A) to vary pH-modifying agent 12A (which in the FIG. 1B embodiment may comprise varying the amount and/or composition of gas 112A) administered to patient P via interface 114. For example, controller 15 may use control signals 15A to control gas source 112 and/or gas mixer 113 to vary the partial pressure of CO₂ in gas 112A and/or to vary the partial pressure of CO₂ and O₂ in gas 112A. It is noted that in addition to impacting pH, the level of circulating CO₂ in a patient is also positively correlated with blood vessel dilation (which in turn is positively correlated with blood flow and negatively correlated with blood pressure) and, consequently, CO₂ can be administered to control blood flow in patient P independently of whether the CO₂ impacts pH. In such circumstances, pH-adjusting actuator(s) 12 of FIGS. 1A and 1B may be considered a vasculature-impacting actuator(s).

FIG. 1C illustrates a blood flow control apparatus 210 according to another example embodiment of the invention. Apparatus 210 differs from apparatus 10, 110 described above in that pH-adjusting actuator(s) 12 of apparatus 210 comprise a drug delivery system 212. Drug delivery system 212 is controlled by controller 15 (using control signals 15A) to vary pH-modifying agent 12A (which in the FIG. 1B may comprise varying the dosage of one or more pH-modifying drugs 212A) administered to patient P via interface 214. For example, controller 15 may use control signals 15A to control drug delivery system 212 to control a dosage of a carbonic anhydrase inhibiting drug, such as acetazolamide and/or the like. It is noted that in addition to impacting pH, circulating drug levels of particular vasculature-impacting drugs (e.g. sodium nitroprusside and/or the like) in a patient are also positively correlated with blood vessel dilation (which in turn is positively correlated with blood flow and negatively correlated with blood pressure) and, consequently, such vasculature-impacting drugs can be administered to control blood flow in patient P independently of whether such vasculature-impacting drugs impact pH. In such circumstances, pH-adjusting actuator(s) 12 of FIGS. 1A and 1C may be considered a vasculature-impacting actuator(s) and pH-modifying drug 212A (of FIG. 1C) may be considered a vasculature-impacting drug.

In some embodiments, controller 15 may use control signals 15A to control both a drug delivery system (like drug delivery system 212 of apparatus 210) and a gas source or gas mixer (like gas source 112 and/or gas mixer 113 of apparatus 110) to administer a variety of pH-modifying agents 12A to patient P via a variety of interfaces 14.

### Examples of Gas Source 112 and Interface 114

As discussed above, in some embodiments (e.g. in apparatus 110 of FIG. 1B), pH-adjusting actuators 12 comprise a gas source 112 and/or mixer 113. In some embodiments gas source 112 comprises a gas mixer 113 that operates to mix plural gases (e.g. oxygen, carbon dioxide and nitrogen) to yield a pH-modifying agent 12A in the form of a pH-modifying gas 112A. Controller 15 may deliver control signals 15A that cause gas mixer 113 to vary the relative amounts (e.g. partial pressures) of the plurality of gases in gas 112A that is administered to patient P via interface 114. Gas mixer 113 may, for example be digitally controlled using control signals 15A.

In some embodiments, gas source 112 (and/or interface 114) comprises a recirculation valve that is adjustable (using control signals 15A) to variably mix exhaled breath from patient P with gas 112A being inspired by patient P. The exhaled breath may have a higher partial pressure of CO₂ than gas 112A such that the partial pressure of CO₂ in gas being breathed in by patient P may be increased by opening the recirculation valve to increase a proportion of exhaled breath in the gas being inspired by patient P.

In some embodiments gas source 112 comprises a source of CO₂ (e.g. that is controllable by control signals 15A). The CO₂ may be delivered (e.g. by interface 114) so that it mixes with gas being inhaled by patient P. For example the CO₂ may be delivered into a nose tube, a ventilator or some other suitable interface 114. In such embodiments controller 15 may be operable (using control signals 15A) to vary a flow rate (or pressure) of the CO₂ being supplied to patient P from gas source 112.

In some embodiments gas source 112 comprises a source of dissolved CO₂ and/or another chemical species (such as carbonate or bicarbonate) that is in equilibrium with CO₂ in the patient's blood) used together with an interface 114 that delivers the dissolved CO₂ or other chemical species into patient P's bloodstream (e.g. by way of an arterial catheter). Controller 15 may use control signals 15A to alter the circulating carbon dioxide levels in patient P by varying delivery of the dissolved CO₂ and/or other chemical species to the bloodstream of patient P.

In some embodiments gas source 112 comprises concentrators that selectively concentrate one or more gases present in air (e.g. CO₂ and/or O₂). In such embodiments the composition of gas 112A delivered to patient P may be varied by controller 15 (using control signals 15A), for example, by selectively mixing gas output by a concentrator (that has a higher concentration of CO₂) with: air that has a relatively lower concentration of CO₂ and/or a mixture of gases (e.g. N₂ and O₂) that contains no CO₂ or reduced CO₂.

In some embodiments gas source 112 combines features of two or more of the above.

In some embodiments, interface 114 may comprise a mouthpiece, nose tube, respirator, ventilator, anaesthetic machine or other means for delivering gas 112A for inspiration to patient P.

In some embodiments interface 114 comprises an arterial catheter and a pump configured to deliver dissolved CO₂ or other chemical species by way of the arterial catheter.

### Examples of Drug Delivery System 212 and Interface 214

In some embodiments, drug delivery system 212 and/or interface 214 may comprise any suitable system for controlling a dosage of a pH-modifying agent 12A (e.g. one or more carbonic anhydrase inhibiting drugs 212A) delivered to patient P. Such drug delivery systems 212 and/or interfaces 214 may be suitable for delivering drugs locally or predominantly locally to CNS regions and/or cardiac regions. Suitable drug delivery systems 212 and/or interfaces 214 comprise implantable or external drug delivery pumps, drug-eluding arrays (e.g. microneedle arrays), drug-eluding stents (e.g. for insertion into coronary arteries) and/or other drug-eluding devices, IV infusing devices, arterial catheters (e.g. inserted into coronary arteries, and/or the like. Drug delivery system 212 and/or interface 214 may interact with patient P is a variety of ways, including percutaneous, epidural, etc.

### Example Controller 15

Controller 15 may comprise an electronic circuit or system that is operative to generate output signal(s) 15A for controlling pH-adjusting actuators 12 based on one or more input signals 16A from sensor(s) 16. Controller 15 may, for example comprise a data processor configured by suitable software instructions to generate output signal(s) 15A from input signal(s) 16A.

Other additional or alternative constructions of controller 15 are also possible. For example in addition to or in the alternative to a data processor that is configured by software, controller 15 may comprise configurable electronics (e.g. a field programmable gate array (FPGA)), a trained neural network, an analog feedback controller circuit etc.

For example, controller 15 may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

### Example Sensors 16

Sensors 16 monitor one or more parameters that are relevant to CNS/cardiac region blood flow and/or blood pressure. For example, sensors 16 may include sensors that monitor one or more of:
blood flow in the region of a CNS injury or CNS region (e.g. spinal cord blood flow or brain blood flow) or in the cardiac region (e.g. heart blood flow or coronary artery blood flow);
spinal cord perfusion pressure;
coronary perfusion pressure;
blood oxygenation in the region of a CNS injury or CNS region (e.g. spinal cord or brain blood O₂ levels) or in the cardiac regions (e.g. heart blood or coronary artery O₂ levels);
blood CO₂ levels in the CNS/cardiac region;
blood velocity in the CNS/cardiac region;
tissue oxygenation/de-oxygenation in the CNS/cardiac region;
hemoglobin oxygenation/de-oxygenation in the CNS/cardiac region;
total oxygenation index in the CNS/cardiac region;
blood pH level in the CNS/cardiac region;
temperature in the CNS/cardiac region.

Sensors 16 may operate according to suitable physical principles. For example, sensors 16 may include one or more of:
near infrared sensors (NIRS);
ultrasound sensors (e.g. Doppler ultrasound blood flow sensors);
infrared temperature sensors;
thermometers based on electronic devices (e.g. thermistors);
intravascular sensors;

Sensors 16 may include sensors that measure other effects of operation of apparatus 10, 110, 210. For example, sensors 16 may include sensors operative to measure one or more of:
composition of gas 112A;
blood CO₂ levels and/or O₂ levels (e.g. Pa_{CO2} levels and/or Pa_{O2} levels)
blood pressure;
body temperature;
other markers.

Sensors 16 may be configured to monitor cardiovascular parameters at or near the site of a CNS injury or in the cardiac region. For example, sensors 16 may comprise non-invasive sensors (e.g. transdermal ultrasound sensors, transdermal near infrared sensors (NIRS)) and/or sensors designed to be implanted or placed by surgery).

In some embodiments sensors 16 may be located away from the CNS region or cardiac region. For example, blood gas levels, blood chemistry, blood pressure etc. may be monitored by one or more sensors 16 placed at a convenient location such as the radial artery in addition to or as an alternative to sensors 16 located to sense conditions at or close to the CNS region or cardiac region.

In an example implementation for treating an SCI, sensors 16 may include one or more sensors that directly or indirectly measure spinal cord perfusion pressure at or near the site of the SCI. As another example, in an implementation where a brain injury is being treated, sensors 16 may include transcranial sensors (e.g. transcranial NIRS sensors and/or transcranial Doppler ultrasound sensors) that monitor blood flow and/or oxygen levels at or near the site of the brain injury. In an example implementation where a cardiac condition is being treated, sensors 16 may include one or more sensors that directly or indirectly measure coronary perfusion pressure at or near the heart.

### Example Control Method 20

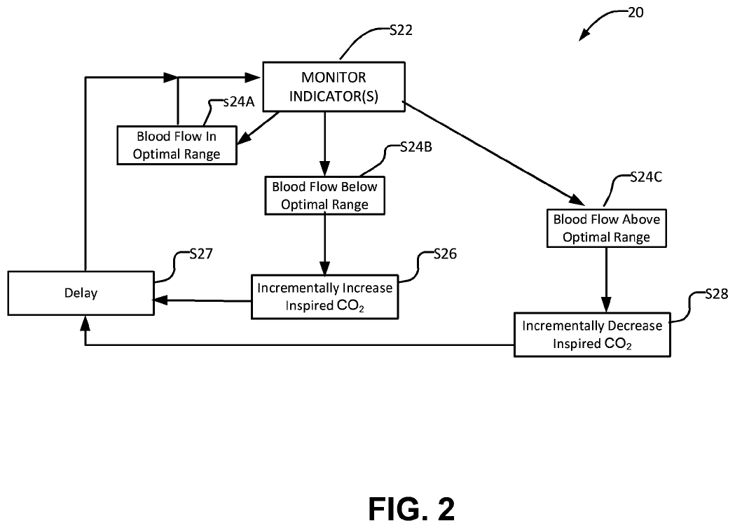

FIG. 2 is a flow chart illustrating an example control method 20 that may be implemented by controller 15 of the FIG. 1B apparatus 110 according to a particular embodiment in the case of a CNS injury. In step S22, one or more value(s) of an input (sensed) parameter or “indicator” is/are measured. Such an indicator may be measured or sensed by sensors 16 and may be provided to controller as sensor signal(s) 16. For example the measured input parameter(s) may directly or indirectly indicate a characteristic of interest (e.g. spinal cord blood flow, spinal cord perfusion pressure, coronary artery blood flow, coronary perfusion pressure and/or the like).

In some embodiments, step S22 involves averaging the value(s) of the measured input parameter(s) (e.g. sensor signals 16A) over suitable period(s) of time (e.g. a time in the range of 10 to 60 seconds such as about 30 seconds) using a moving average filter and/or the like.

Step S24 determines whether the sensed value(s) determined in step S22 indicate that a characteristic of interest (e.g. spinal blood flow, spinal perfusion pressure, coronary artery blood flow, coronary perfusion pressure and/or the like) is below (S24B), within (S24A) or above (S24C) an optimal or desired range for the characteristic of interest (see desired range of characteristic of interest 11 in FIG. 1B). Step S24A loops back to step S22 in the case where the characteristic of interest is within a desired range.

If step S24 determines that the characteristic of interest is below the optimal range, then method proceeds via step S24B to step S26. In the illustrated embodiment of FIG. 2, where method 20 is used to control apparatus 110 of FIG. 1B used in the case of a CNS injury, step S26 involves generating control signals 15A which will cause an incremental increase in an amount of inspired CO2 (e.g. an increment of about 2 mmHg). This step S26 increase in CO2 may cause a corresponding decrease in pH and a corresponding increase in blood flow to the region of the CNS injury (or the cardiac region as the case may be).

In the general case (e.g. of apparatus 10 of FIG. 1A), step S26 may involve generating control signals 15A which will cause pH-adjusting actuators 12 to make an incremental change in pH-modifying agent 12A in such a manner as to attempt to cause a corresponding decrease in pH and a corresponding increase in blood flow to the region of the CNS injury (or the cardiac region as the case may be). In the case of apparatus 210 of FIG. 1C), step S26 may involve generating control signals 15A which will cause drug delivery system 212 to make an incremental change in pH-modifying drug(s) 212A administered to patient P in such a manner as to attempt to cause a corresponding decrease in pH and a corresponding increase in blood flow to the region of the CNS injury (or the cardiac region as the case may be).

Subsequently, after a short delay in step S27, method 20 loops back to step S22.

If step S24 determines that the characteristic of interest is above the optimal range, then method proceeds via step S24C to step S28. In the illustrated embodiment of FIG. 2, where method 20 is used to control apparatus 110 of FIG. 1B used in the case of a CNS injury, step S28 involves generating control signals 15A which will cause an incremental decrease in an amount of inspired CO2 (e.g. an decrement of about 2 mmHg). This step S28 decrease in CO2 may cause a corresponding increase in pH and a corresponding decrease in blood flow to the region of the CNS injury (or the cardiac region as the case may be).

In the general case (e.g. of apparatus 10 of FIG. 1A), step S28 may involve generating control signals 15A which will cause pH-adjusting actuators 12 to make an incremental change in pH-modifying agent 12A in such a manner as to attempt to cause a corresponding increase in pH and a corresponding decrease in blood flow to the region of the CNS injury (or the cardiac region as the case may be). In the case of apparatus 210 of FIG. 1C), step S28 may involve generating control signals 15A which will cause drug delivery system 212 to make an incremental change in pH-modifying drug(s) 212A administered to patient P in such a manner as to attempt to cause a corresponding increase in pH and a corresponding decrease in blood flow to the region of the CNS injury (or the cardiac region as the case may be).

Subsequently, after a short delay in step S27, method 20 loops back to step S22.

Apparatus 10 may be applied to maintain a sufficiently high blood flow in the neighborhood of a CNS region (in the event of a CNS injury) or cardiac region (in the event of a cardiac condition). Apparatus 10 may also be applied to temporarily depress blood flow to a neighborhood of interest (for example to assist in controlling bleeding or to allow application of a treatment that may benefit from reduced blood flow).

In some embodiments, apparatus 10 is operative to use blood oxygen levels as well as circulating carbon dioxide levels to control blood flow in a region of interest (e.g. in a region of a CNS injury or a cardiac region). For example, a combination of blood flow and blood oxygen content may be controlled to ensure optimal oxygen delivery to the region of interest. By controlling blood oxygen levels in conjunction with controlling blood flow at the site of an injury, it may be possible to maintain sufficient delivery of oxygen to the site of injury while blood flow to the site of injury is adjusted up and down. For example, having regard to apparatus 110 of FIG. 1B, the oxygen content of gas 112A may be adjusted based on characteristics of interest such as: blood oxygen levels, blood flow at or near the site of injury and/or perfusion pressure at the site of injury.

Example User Interface Controls

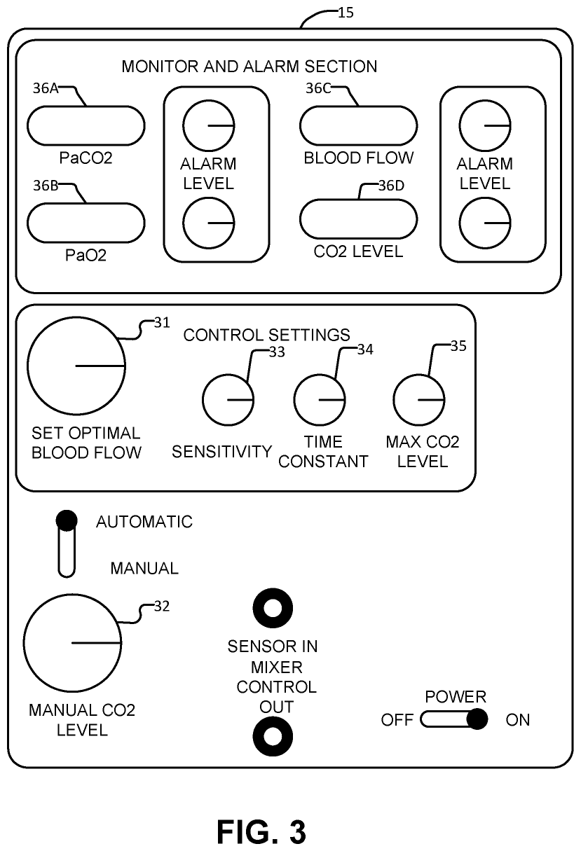

Apparatus 10 may include various controls that may be used to adjust the operation of apparatus 10. FIG. 3 shows an illustrative example of a controller 15 that includes various controls. For example, apparatus 10 may include:
a control 31 that allows the optimum range for a controlled characteristic of interest 11 (e.g. spinal cord blood flow, spinal perfusion pressure, coronary artery blood flow, coronary perfusion pressure and/or the like) to be set.
a control 32 that allows the control signal(s) 15A to be manually controlled (e.g. to override the automatic control by controller 15 of pH-adjusting actuators 12 and the corresponding characteristics of the pH-modifying agent 12A being delivered to patient P.
a control 33 that adjusts the sensitivity of controller 15 to changes in sensor input signals 16A from sensor(s) 16.
a control 34 that adjusts how quickly controller 15 responds to changes in sensor input signals 16A from sensors 16.
a control 35 that sets limits (e.g. maxima or minima) for one or more parameters effected by control signals 15A, such as the partial pressures of oxygen and/or carbon dioxide in gas 112A n the case of apparatus 110) or the parameters of pH-modifying agent 12A (in the case of apparatus 10) o the parameters of pH-modifying drug(s) 212A (in the case of apparatus 210).

Apparatus 10 may include various displays 36 that communicate information such as proper operation of apparatus 10, the status of patient P, values for outputs of one or more sensors 16, warning signals (e.g. warning that a sensor signal 16A indicates a value of a physiological parameter or other parameter outside of a corresponding range or that the characteristic of interest 11 (e.g. CNS blood flow, perfusion pressure, cardiac blood flow, coronary perfusion pressure and/or the like) is outside of a corresponding range) and the like. FIG. 3 of the illustrated embodiment includes displays 36A, 36B which respectively display CO2 and O2 levels and displays 36C, 36D which respectively display blood flow measurements and CO2 levels. Each display 36 in FIG. 3 has a corresponding control for setting a corresponding alarm limit. Displays of other additional or alternative parameters (e.g. drug concentrations and/or the like) and other additional or alternative alarm limits may be provided.

### Example Application of Apparatus 10, 110, 210

Apparatus 10, 110, 210 may, for example be used to stabilize conditions at the site of an injury such as an SCI, traumatic brain injury (TBI), in a cardiac region (e.g. in the heart or in one or more coronary arteries) and/or the like. Apparatus 10, 110, 210 may help to maintain blood flow and oxygenation of the spinal cord, the heart or other injured or non-optimally performing tissues. For example, apparatus 10, 110, 210 may be used in an intensive care unit, acute, or step-down unit of a hospital during the neurogenic and acute phase of spinal cord injury or cardiac event. In some examples of such applications, apparatus 10, 110, 210 may operate to stabilize blood flow through the spinal cord and prevent ischemia that causes secondary damage to the spinal cord in the acute phase of injury.

For example, in the case of apparatus 110, where a patient presents with a spinal cord injury, a team may immediately physically stabilize the site of the injury and apply suitable sensors 16 (e.g. a Doppler blood flow monitor). The patient may be connected to an interface 14 to receive gas 112A from gas source 112. Apparatus 110 may then control the composition of gas 112A as described above to maintain adequate blood flow in and about the site of the injury.

The invention may also be provided in the form of a program product. The program product may comprise any non-transitory medium which carries a set of computer-readable instructions which, when executed by a data processor, cause the data processor to execute a method of the invention (e.g. a control process performed by controller 15). Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

### Experimental Results

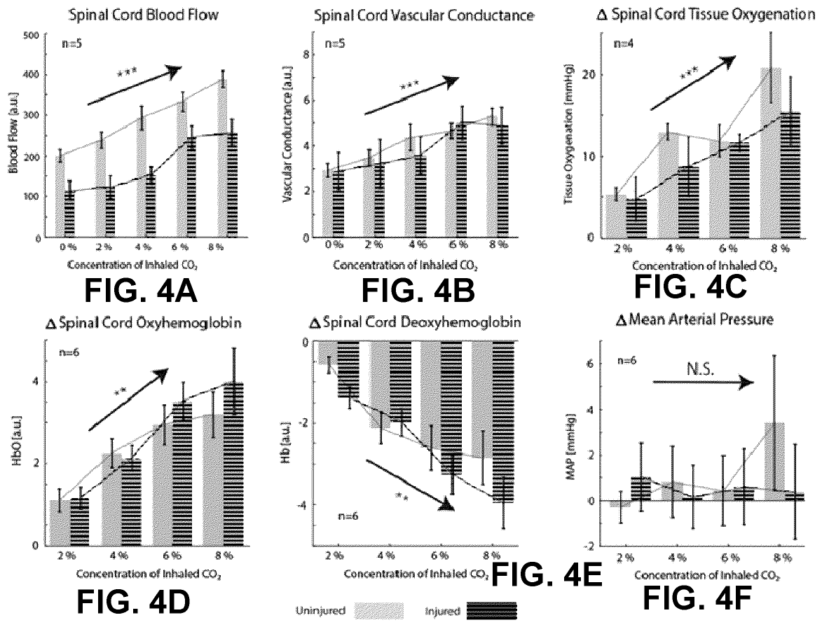

FIGS. 4A-F show various plots presenting the correlation between circulating carbon dioxide level and various physiological parameters in an experimental setting. For example, FIG. 4A shows the relationship between spinal cord blood flow and concentration of inhaled CO₂ in injured and uninjured specimens. As evident from FIG. 4A, there is a positive correlation relationship between spinal cord blood flow and concentration of inhaled CO₂ for both injured and uninjured specimens.

Similarly, FIGS. 4B-D show a positive correlation between concentration of inhaled CO₂ and spinal cord vascular conductance, change in spinal cord tissue oxygenation, and change in spinal cord oxyhemoglobin respectively. FIG. 4E shows that concentration of inhaled CO₂ has a negative correlation with change in spinal cord deoxyhemoglobin. This is consistent with the result presented in FIG. 4D. Finally, FIG. 4F shows that the mean change in arterial pressure in injured specimens are maintained in relation to change in concentration of inhaled CO₂.

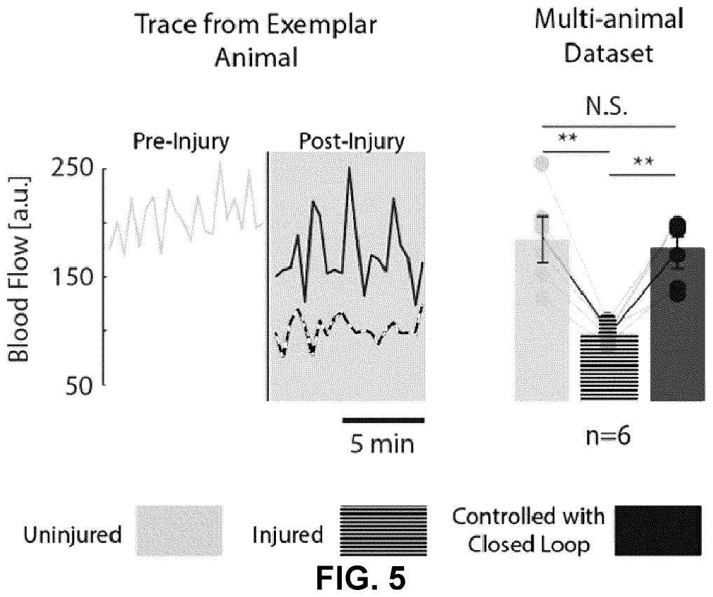

FIG. 5 is a plot tracing blood flow of uninjured test animals, injured test animals and injured test animals treated by the method in FIG. 2 over a period of time in an experimental setting. FIG. 5 shows that blood flow in the test animal is meaningfully increased by controlled closed loop method as described herein in this application to be comparable to blood flow of the test animal in an uninjured state.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and.
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a component (e.g. a gas source, mixer, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein have discrete components and features which may be readily separated from or combined with the features of any other described embodiment(s) without departing from the scope of the present invention.

Any aspects described above in reference to the apparatus may also apply to methods and vice versa.

Any recited method can be carried out in the order of events recited, or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. Apparatus for stabilizing blood flow, the apparatus comprising:
a pH-adjusting actuator operative to deliver a pH-modifying agent to a patient;
a controller connected to control the pH-adjusting actuator to alter a level at which the pH-modifying agent is delivered to the patient in response to an output signal from a sensor, the sensor operative to monitor blood flow;
wherein the controller is configured to cause the pH-adjusting actuator to modify the level at which the pH-modifying agent is delivered to the patient in a manner which causes the blood pH in the patient to decrease in response to the sensor output signal indicating a decrease in the blood flow.

2. The apparatus according to claim 1 wherein the pH-adjusting actuator comprises a gas source and the pH modifying agent comprises CO₂ such that the gas source is operative to deliver CO₂ to the patient.

3. The apparatus according to claim 2 wherein the controller is configured to increase the level at which the CO₂ is delivered to the patient in response to the sensor output signal indicating a decrease in the blood flow.

4. The apparatus according to claim 2 wherein:
the gas source comprises a gas mixer operative to output a mixed gas, a source of CO₂ and a source of other breathable gases;
wherein the controller is operable to control the gas source by controlling the gas mixer to vary a ratio of CO₂ from the source of CO₂ to other breathable gases from the source of other breathable gases in the mixed gas and the apparatus comprises an interface configured to supply the mixed gas to the patient for breathing.

5. The apparatus according to claim 2 wherein the controller is configured to:
average the output signal from the sensor over a time period;
process the averaged output signal to determine whether the blood flow is below, within, or above an optimal range;
in response to the blood flow being below the optimal range increase the level at which the CO₂ is delivered until the blood flow is within the optimal range; and
in response to the blood flow being above the optimal range decrease the level at which the CO₂ is delivered until the blood flow is within the optimal range.

6. The apparatus according to claim 5 wherein the controller is configured to increase the level at which the CO₂ is delivered by incrementally increasing the level and pausing between increments.

7. The apparatus according to claim 1 wherein the pH-adjusting actuator comprises a drug-delivery system and the pH-modifying agent comprises a pH-modifying drug, the drug-delivery system operable to deliver the pH-modifying drug to the patient.

8. The apparatus according to claim 7 wherein the pH-modifying drug comprises a carbonic anhydrase inhibiting drug.

9. The apparatus according to claim 8 wherein the carbonic anhydrase inhibiting drug comprises acetazolamide.

10. The apparatus according to claim 7 wherein the controller is configured to increase a dosage at which the pH modifying drug is delivered to the patient in response to the sensor output signal indicating a decrease in the blood flow.

11. The apparatus according to claim 7 wherein the drug-delivery system comprises a drug-delivery pump.

12. The apparatus according to claim 7 wherein the drug-delivery system comprises a drug-eluding device.

13. The apparatus according to claim 7 wherein the controller is configured to:
average the output signal from the sensor over a time period;
process the averaged output signal to determine whether the blood flow is below, within, or above an optimal range;
in response to the blood flow being below the optimal range increase the level at which the pH-modifying drug is delivered until the blood flow is within the optimal range; and
in response to the blood flow being above the optimal range decrease the level at which the pH-modifying drug is delivered until the blood flow is within the optimal range.

14. The apparatus according to claim 13 wherein the controller is configured to increase the level at which the pH-modifying drug is delivered by incrementally increasing the level and pausing between increments.

15. The apparatus according to claim 1 wherein the sensor comprises a Doppler ultrasound blood flow sensor.

16. The apparatus according to claim 1 wherein the sensor is operational to monitor at least one of: spinal cord perfusion pressure and coronary perfusion pressure.

17. The apparatus according to claim 1 wherein the sensor comprises a near infrared sensor.

18. The apparatus according to claim 1 wherein the controller is configured to execute a feedback control algorithm.

19. A controller for stabilizing blood flow, the controller comprising:
an input for receiving a sensor output signal indicative of blood flow in a patient;
an output for outputting a control signal for a pH-adjusting actuator operative to deliver a pH-modifying agent to a patient;
a processor configured to execute a feedback control algorithm, the feedback control algorithm operative to, in response to the blood flow being lower than an optimal range, set the control signal to cause the pH-adjusting actuator to modify an output level of the pH-modifying agent for delivery to the patient.

20. The controller according to claim 19 wherein the pH-adjusting actuator comprises a gas source and the pH-modifying agent comprises CO₂, the gas source operative to deliver CO₂ to the patient.

21.-51. (canceled)
